(19) United States
(12) Patent Application Publication
(10) Pub. No.: US 2024/0255487 A1
(43) Pub. Date: Aug. 1, 2024

(54) REAL TIME DETECTION OF SOLIDS CONTENT IN AQUEOUS COLLOIDAL DISPERSIONS SUCH AS OIL SANDS TAILINGS USING MICROWAVE SENSORS

(71) Applicant: KEMIRA OYJ, Helsinki (FI)

(72) Inventors: Sampsa GREUS, Espoo (FI); Mehrdad HESAMPOUR, Espoo (FI)

(21) Appl. No.: 18/566,126

(22) PCT Filed: May 27, 2022
(86) PCT No.: PCT/US2022/031237

§ 371 Date: Dec. 1, 2023

Related U.S. Application Data
(60) Provisional application No. 63/195,430, filed on Jun. 1, 2021.

(30) Foreign Application Priority Data
Oct. 20, 2021 (FI) 20216087

(51) Int. Cl. G01N 33/28; C08L 95/00; C10C 3/14; C10G 1/04

(52) U.S. Cl. CPC G01N 33/2823; C08L 95/00; C10C 3/14; C10G 1/047

(57) ABSTRACT

Industrial methods which utilize microwave-based sensors to detect in real-time the total solids content of aqueous solid colloidal dispersions such as oil sands tailings streams are provided. Optionally, these microwave-based sensors may be utilized in combination with automatic cleaning systems or filters, which prevent sensor fouling and allow for extended sensor use without manual cleaning. The output signals from the microwave sensor are used to adjust desired process parameters, e.g., the dosage of chemical additives and/or to maintain total solids within specified limits.

### RELATED APPLICATIONS

The present invention claims priority to U.S. Provisional Application No. 63/195,430 filed on Jun. 1, 2021 and Finnish application No. 20216087 filed on Oct. 20, 2021, the contents of both of which are incorporated by reference in their entireties.

### FIELD OF THE INVENTION

The present invention relates to a method for real-time monitoring of total solids or total suspended solids content of oil sands tailings streams. Also, the invention relates to industrial systems for effecting such methods.

### BACKGROUND OF THE INVENTION

Bituminous sands, also referred to as oil sands, are a type of petroleum deposit. Oil sands typically contain naturally occurring mixtures of sand, clay, water, and a dense, extremely viscous form of petroleum technically referred to as bitumen, or colloquially “tar” due to their similar appearance, odor, and color. Oil sands may be found in large quantities in many countries throughout the world, most abundantly so in Canada and Venezuela. Oil sand deposits in northern Alberta in Canada (Athabasca oil sands) are thought to contain approximately 1.6 trillion barrels of bitumen.

Since bitumen flows very slowly, if at all, the bituminous sands may be extracted by strip mining or made to flow into wells by in situ techniques that reduce the viscosity, such as by injecting steam, solvents, and/or hot air into the sands. These processes may use more water and may require larger amounts of energy than conventional oil extraction. After mining operations are completed, the oil sands are crushed to break down large clumps and additional hot water is added to form a slurry of sand, clay, bitumen, and water that can be pumped to an extraction plant, where bitumen is separated from the other components. These leftover components collectively constitute tailings.

Water-based oil sand extraction processes generally include ore preparation, extraction, and tailings treatment stages wherein a large volume of solids-laden aqueous tailings may typically be produced. Oil sands tailings are a mixture of water, sand, fine silts, clay, residual bitumen and lighter hydrocarbons, inorganic salts and water-soluble organic compounds. These tailings may generally be referred to as oil sands process tailings, or oil sands tailings. Tailings may require solid-liquid separation in order to reduce the total suspended solids in the tailings to within specific limits so that the water may be efficiently recycled and used in subsequent extraction processes.

In many processes, these oil sands tailings are pumped to large settling ponds or tailings ponds. In tailings ponds, the process water, unrecovered hydrocarbons and minerals generally settle naturally to form different strata. The upper stratum is usually primarily water that may be recycled as process water to the extraction process. The lower stratum generally contains the heaviest materials, mostly sand, which settle to the bottom of the pond. The middle stratum, often referred to as “mature fine tailings” (“MFT”), generally includes water and hydrophilic and biwetted ultrafine solids, mainly clays and other charged silicates and metal oxides, that tend to form stable colloids in water and exhibit a very slow settling and dewatering behavior, resulting in tailing ponds that may take several years to manage.

The composition of mature fine tailings tends to be highly variable. Near the top of the stratum the mineral content may be about 10% by weight and over time may consolidate and comprise up to 50% by weight of the materials contained at the bottom of the stratum. Overall, mature fine tailings may have an average mineral content of about 30% by weight. While fines may generally be the dominant particle size fraction in the mineral content, the sand content may be 15% by weight of the solids and the clay content may be up to 100% by weight of the solids, reflecting the oil sand ore and extraction process. Additional variation may result from the residual hydrocarbon which may be dispersed in the mineral or may segregate into mat layers of hydrocarbon. The mature fine tailings in a pond may not only contain a wide variation of compositions distributed from top to bottom of the pond, but also may contain pockets of different compositions at random locations throughout the pond. Additionally, mature fine tailings generally behave as a fluid-like colloidal material.

In order for water from tailing ponds to be efficiently recycled and used in subsequent extraction processes, material from the upper layers of tailing ponds must be effectively dewatered so that total suspended solids may be removed. The slow settling of fine (<45 μm) and ultrafine clays as well as the large demand of water during oil sand extraction process have promoted research and development of new technologies to modify the water release and to improve settling characteristics of tailings streams.

Centrifuges are typically used for dewatering of oil sands tailings. In this process dewatering is assisted by addition of anionic polymer to process stream feeds to the centrifuges. The outlet streams of centrifuge form a cake, with higher dry solid content, and aqueous stream, often called centrate. Most solids transfer into the cake but some clays and ultra-fine solids (<2 μm) are often challenging to capture and in many instances, may remain suspended in the centrate, which will be recycled back to the extraction process. These solids may be detrimental to bitumen recovery, and as such, optimizing dosage and composition of polymer additives to remove the fines from the water during tailings treatment is of general importance.

Monitoring total solids or total suspended solids in the centrate gives indication of centrifuge performance and indicates whether centrate water is clean enough to safely reuse or discharge. Without the ability to obtain consistent online measurements in real-time, it is difficult to properly dose polymeric additives, such as coagulants and flocculants, which are required for maintaining on-spec performance. Obtaining consistent online measurements in real-time from process streams has not previously been successful due to the physical properties of said process streams, which poses difficulties for utilizing typical water treatment and processing industry sensors, e.g., optical turbidity and total suspended solids (TSS) sensors.

Among the known methods used for analyzing the composition of the extracted oil sands are near infrared (NIR) and radio spectrometry. Both are used to assess the concentration of constituents in oil sands where the reflectance spectra range from 1100 nm to 2500 nm and the specific oil sands components have specific wavelengths, for example 1400 nm for water, 1720 nm for oil, 2200 nm for kaolinite. Another method used in the mining or oil sand industry is the spectroscopic analysis of oil sands, which uses the signals containing information about the images of the ore sample to create a real time ore grade visualization including a composite overlay image of the ore sample.

Also, nuclear magnetic resonance pulse spectrometry has been used to analyze oil sands composition by initially saturating the magnetization of the oil sand sample and then subjecting the samples to a sequence of radio-frequency pulses optimized for the measurement of bitumen and water in the sample. The amount of bitumen and water is determined based on a partial least squares optimization based chemometric model.

The oil content in oil sands has also been measured using an acoustic technique, by observing the nonlinear dissipation phenomenon that is generated by the sound wave spreading in the oil sands.

There are also other methods for analyzing materials extracted from an earth formation. Prompt gamma neutron activation analysis (PGNAA) is one such method that is generally used to determine metal contents of ores. PGNAA has also been used to detect a clay parameter indicating, for example, a weight percentage of clay particles in an oil sand tailings stream.

In another method which involves using pulse neutron spectroscopy, the composition of the hydrocarbon material in the material extracted from an earth formation can be calculated based on the at least one gamma ray spectrum detected at the pulse neutron spectroscopy tool which emits a plurality of pulses of high-energy neutrons into the portion of the hydrocarbon material diverted and stored into a container.

Notwithstanding the foregoing, improved methods for analyzing oil sands composition, are desired. In particular, there is a need for systems and methods for real-time, on-stream analysis of oil sand tailings compositions that can measure the total solids or total suspended solids content accurately and in a continuous manner.

The present invention seeks to address such problems by providing novel methods and systems that facilitate real-time monitoring of total suspended solids in oil sands tailings streams including, but not limited to, total solids or total suspended solids in the release water and/or feed of a centrifugation, thickening, filtration, hydrocyclone, or inline flocculation process.

### SUMMARY OF THE INVENTION

The present invention provides novel industrial methods and industrial systems used in such industrial methods that use microwave-based sensors for real-time monitoring of total suspended solids in aqueous colloidal dispersions, preferably oil sands tailings, for use in methods which are used to treat the solid aqueous colloidal dispersions, preferably oil sands tailings, in order to separate the water from the solids contained therein. Such microwave-based sensors are used in particular to monitor solid content in oil sands tailings streams including, but not limited to, total solids or total suspended solids in the release water and/or feed of a centrifugation, thickening, filtration, hydrocyclone, or inline flocculation process centrate and/or feed to a centrifuge.

In general the output signals from the microwave sensor and optionally other sensors are then used to determine in real time whether any parameters in the method should be altered, e.g., the output signals may be entered into dosing programs which are used to adjust the dosage of chemicals or other parameters used in the industrial process being conducted, e.g., the dosages of polymers such as flocculants or coagulants typically used in oil sands treatment, and/or the output signals are used in order to maintain total solids or total suspended solids within specified limits.

Based on the foregoing, in one aspect, the present invention in general relates to an industrial process for treating an aqueous colloidal dispersion that comprises the following steps:
a) separating solids from water comprised in an aqueous colloidal dispersion, that comprises water and solids, and
b) detecting and/or monitoring in real time the solids content (suspended solids content) of an aqueous colloidal dispersion in real time with a microwave sensor, optionally wherein said method further includes detecting in real time one or more other parameters with sensors, e.g., (i) pH, (ii) particle size, (iii) temperature, (iv) pressure, (v) solid-liquid separation rate (vi) influx or efflux rate of colloidal dispersion, (vii) amount of free or dissolved air or CO₂ in the detected sample or any combination of the foregoing.

In another aspect, the present invention in general relates to an industrial process for treating an aqueous colloidal dispersion that comprises the above steps a) and b), wherein said process further includes a step c) wherein the detected amount of solids alone or optionally in conjunction with one or more other parameters detected in real time, is used to determine whether one or more parameters should be modified during the process, optionally wherein the detected solids amount is input to a controller where further optionally, based on a dosing algorithm, output signal is sent to dosing pumps, wherein said other detected parameters include (i) temperature, (ii) pH, (iii) pressure, (iv) the introduction of a chemical or other moiety into the system such as a coagulant, flocculant, biocide, enzyme, or polymer, (v) the adjustment of the dosage of any of the foregoing, (vi) the speed or velocity of the influx or efflux of the aqueous colloidal dispersion through the system, (vii) solid-liquid separation rate (e.g., g-force or rpm), or any combination of the foregoing, wherein optionally said controllers provide for one or more of said parameters to be adjusted based on the detected solids content alone or in association with another detected parameter.

While the methods are broadly applicable to any industrial process wherein aqueous colloidal dispersions are produced and treated typically the aqueous colloidal dispersions comprise oil sands tailings or another aqueous colloidal dispersion comprising bitumen or other dark solids and/or it comprises an optically turbid aqueous colloidal dispersion comprising dispersed or colloidal solids.

In a preferred aspect, the present invention relates to an industrial process as afore-described, wherein said process further includes a step c) wherein the detected amount of solids is used to determine whether one or more parameters should be modified during the process, wherein said parameters optionally include (i) temperature, (ii) pressure, (iii) the introduction of a chemical or other moiety introduced into the system such as a coagulant, flocculant, biocide, enzyme, polymer, et al., or (iv) the adjustment of the dosage of any of the foregoing, (v) the speed or velocity of the influx or efflux of the aqueous colloidal dispersion through the system, (vi) solid-liquid separation rate, (vii) pH, (viii) particle size, or any combination of the foregoing.

In exemplary aspects the treated aqueous colloidal dispersion comprises oil sands tailings or another aqueous colloidal dispersion comprising bitumen or other dark solids and/or it comprises another optically turbid aqueous colloidal dispersion comprising dispersed solids.

In preferred exemplary aspects the treated aqueous colloidal dispersion comprises oil sands tailings.

In exemplary aspects, said real time detecting and/or monitoring is effected continuously or periodically as the industrial process is conducted.

In some exemplary aspects, the amount of detected solids in the aqueous colloidal dispersion, e.g., oil sand tailings composition, is used to adjust the dosage of coagulant or flocculant added to the system.

In some exemplary aspects, the amount of detected solids is detected in a centrate of centrifuge, or filtrate of filter press, a sensor for release water from thickeners (i.e., thickener overflow) or any other equipment used in the industrial process, e.g., an oil sands treatment process.

In some exemplary aspects the amount of detected solids is detected during non-mechanical methods of treating tailings, e.g., during inline flocculation of tailings or thin lift deposition tailings treatment processes.

In some exemplary aspects, the amount of solids or suspended solids is detected in the feed or output or is detected in any composition produced or used at any point during a tailings treatment process, wherein such methods Include mechanical and non-mechanical separation methods, e.g., the amount of solids or suspended solids is detected in thickeners, filter press, thin-lift deposition, and the like.

In some exemplary aspects, the amount of solids is detected in the feed to a centrifugation, thickening, filtration, hydrocyclone, or inline flocculation process.

In some exemplary aspects, the process is run under pressure, optionally 0.2-5 bar, preferably 1-3 bar and more preferably to 1.5 to 2.0 bar.

In some exemplary aspects, one or more other parameters are detected in real time, e.g., temperature, pH, pressure, particle size distribution, the speed or velocity of the influx or efflux of the aqueous colloidal dispersion through the system, solid-liquid separation (e.g., centrifugation, filtration, etc.) rate, dosage of one or more chemicals, amount of free or dissolved air in the sample, or any combination of the foregoing.

In some exemplary aspects, said parameters are periodically or continuously detected by use of a system that uses computers/networks to monitor in real time one or more sensors that detect said one or more parameters.

In some exemplary aspects, said parameters which are periodically or continuously detected include any of the following in the oil sands tailings stream or other aqueous solid dispersion: detection of moisture content, detection of pH, detection of elemental composition, detection of sulfur content, detection of iron content, detection of clay amount, detection of magnesium amount, detection of sodium amount, detection of aluminum amount, detection of calcium amount, detection of hydrogen amount, detection of silicon amount, detection of potassium amount, detection of particle size distribution, or any combination of the foregoing.

In some exemplary aspects, the system or method includes the use of one or more gamma detectors or gamma neutron activation analyzers, microwave sensors, pressure sensors, temperature sensors or dissolved air sensors.

In some exemplary aspects, the detected amount of solids or suspended solids Is periodically compared to that of a control sample containing a known solids content as a quality control.

In some exemplary aspects, a prefilter or strainer or other removal means is used to remove large particles or clumps from the centrate stream or other aqueous solid dispersion stream prior to the stream being contacted with the microwave sensor.

In more specific embodiments, e.g., as shown in FIG. 13, a filter cleaner system is provided which removes bitumen residue from the filter.

In some exemplary aspects, the system includes an automatic cleaning system, optionally a water flushing system or a chemical cleaning setup for the sensor.

In some exemplary aspects, the system used to conduct the industrial process is schematically depicted in FIG. 2 or FIG. 13.

In some exemplary aspects the invention permits the usage of reduced amounts of one or more chemicals used in the industrial process, e.g., polymers or non-polymeric chemical additives, e.g., flocculants and coagulants.

In some exemplary aspects the invention provides for the separation of greater amounts of solids from the treated aqueous colloidal dispersion, e.g., oil sands tailings than otherwise equivalent methods which do not include real-time monitoring of solids.

In some exemplary aspects the invention provides for the separation of more water, and/or the recovery of higher purity water, from the treated aqueous colloidal dispersion, e.g., oil sands tailings, than otherwise equivalent methods which do not include real-time monitoring of solids.

In some exemplary aspects the invention provides for the process to be conducted more rapidly than processes conducted without such real time monitoring of solids content.

In some exemplary aspects the invention provides for the process to be conducted at pressures which preclude air bubble formation.

In other exemplary aspects, the invention is directed to an industrial system used in separating water from solids comprised in an aqueous colloidal dispersion that comprises water and solids, optionally oil sands tailings, wherein the system comprises one or more a microwave sensors that detect and/or monitor in real time the solids content of said aqueous colloidal dispersion as said industrial process proceeds.

In some exemplary aspects, the industrial system includes a centrifuge plant, or any materials and apparatus used in mechanical and non-mechanical methods of treating tailings, e.g., such as are used during inline flocculation of tailings or thin lift deposition tailings treatment processes, e.g., the solids content of thickeners, filter press, compositions/materials used in thin-lift deposition, and the like or any oil sands tailings treatment plant may be monitored.

In some exemplary aspects, the industrial system comprises multiple sensors which are connected to computers/networks to determine what is happening in the industrial system in real time.

In some exemplary aspects, the industrial system includes one or more sensors that detect one or more of the following other values in the oil sands tailings stream or other aqueous solid dispersion or the equipment used in the system: moisture content, pH, elemental composition, sulfur content, iron content, clay amount, magnesium amount, sodium amount, aluminum amount, calcium amount, hydrogen amount, silicon amount, potassium amount, particle size distribution, or any combination of the foregoing.

In some exemplary aspects, the industrial system includes one or more gamma detectors or gamma neutron activation analyzers, microwave sensors, pressure sensors, temperature sensors or dissolved air sensors.

In some exemplary aspects, the industrial system is depicted schematically in FIG. 2A, 2B or 13.

In some exemplary aspects, the industrial system comprises a prefilter or strainer or other removal means which is used to remove large particles or clumps from the centrate stream or other aqueous solid dispersion stream prior to the stream being contacted with the microwave sensor.

In other exemplary aspects, the system may include an automatic cleaning system, optionally a water flushing system or a chemical cleaning setup for the sensor.

### BRIEF DESCRIPTION OF THE DRAWINGS

The invention will be described in more detail with reference to appended drawings, described in detail below.

FIG. 1 provides an exemplary picture of a centrate sample with a dark appearance and results of total suspended solids (TSS) analysis with a turbidimeter according to Example 1.

FIG. 2A-B provides exemplary flow charts of material flow (solid lines) and data flow (dashed lines) in industrial processes for treating tailings streams according to Example 2. An exemplary flow chart is shown for one of many possible methods for using a microwave-based sensor to for real-time monitoring of total suspended solids in oil sands tailings streams including, but not limited to, feed and/or exhaust water from solid-liquid separation (FIG. 2A) and centrate and/or feed to centrifuges (FIG. 2B).

FIG. 3 provides an exemplary diagram of one of many possible microwave sensors used for real-time monitoring of total suspended solids in oil sands process streams according to Example 3.

FIG. 4 provides an exemplary diagram and image of a test rig, with component numbers of the diagram (left) corresponding to numbered components in the image (right), utilized for evaluation of real-time microwave sensing of total suspended solids in centrate samples according to Example 4.

FIG. 5 provides a snapshot of the dashboard of a computer/network system referred to by the present application as “Controller/Sensor” which is used to monitor parameters such as temperature, pressure, total suspended solids in centrate streams determined in real-time from a microwave sensor (Dry solid sensor (%)) and other sensors, and parallel laboratory analysis of total dry solids in samples taken manually (Dry solid quick test (%)) according to Example 5.

FIG. 6 provides an exemplary graph of total % solids in centrate stream determined by (i) real-time microwave sensor monitoring and (ii) parallel laboratory analysis of total dry solids in samples taken manually over time according to Example 6.

FIG. 7 provides an exemplary graph correlating real-time microwave sensor data (Solids % (sensor) and lab measurement (Solids % (lab)) according to Example 7.

FIG. 8 provides an exemplary graph correlating real-time microwave sensor data (Solids % (sensor) and lab measurement (Solids % (lab)) across a range of temperatures according to Example 8.

FIG. 9 provides an exemplary graph correlating real-time microwave sensor data (Solids % (sensor)) and lab measurement (Dry solids %) across a range of temperatures, conductivities, and pH values according to Example 9.

FIG. 10 provides an exemplary color map of correlation factors from parallel comparison of real-time microwave sensor data (Solids % (sensor)) and lab measurement (Solids % (lab)) across a range of temperatures and pressures according to Example 10.

FIG. 11 provides an exemplary color map of correlation factors from parallel comparison of real-time microwave sensor data (Solids % (sensor)) and lab measurement (Solids % (lab)) across a range of temperatures, conductivities, and pH values according to Example 11.

FIG. 12A-C provides exemplary images of bitumen blockage in the test rig as detailed in Example 12. FIG. 12A provides an exemplary image of residual bitumen collected from bottom of feed tank in test rig, FIG. 12B provides an exemplary image of a bitumen clump blocking the sensor after running 10 days, and FIG. 12C provides an exemplary image of bitumen blocking the diaphragm pump after running 10 days.

FIG. 13 provides an exemplary flow chart of one of many possible methods for using a microwave-based sensor to for real-time monitoring of total suspended solids in release water lines from a tailings treatment process, wherein a filtration screen is employed as a pretreatment method to remove large particulates and an automatic cleaning system is employed to allow for extended use without manual cleaning according to Example 13.

### DETAILED DESCRIPTION OF THE INVENTION

Before describing the invention, the following definitions are provided. Unless stated otherwise all terms are to be construed as they would be by a person skilled in the art.

### Definitions

As used herein the singular forms “a”, “and”, and “the” include plural referents unless the context clearly dictates otherwise.

The terms “bituminous sands” or “oil sands” refer to a type of petroleum deposit, which typically contains naturally occurring mixtures of sand, clay, water, and a dense, extremely viscous, non-free flowing form of petroleum technically referred to as “bitumen” (or colloquially “tar” due to their similar appearance, odor, and color).

The term “process stream” generally refers to any aqueous fluids or slurries produced during any type of industrial process, for example, an oil or gas extraction or recovery process, waste treatment process, or any portion thereof. An exemplary process stream includes a diluted bitumen product, such as an oil sand slurry, from any phase of the oil sand mining process including recovery, extraction, refining, or waste treatment.

The terms “tailings” and “tailings stream” generally refer to the discarded materials that may be generated in the course of extracting a valuable material from an ore. Generally, any mining or mineral processing operation that uses water to convey or wash materials will typically generate a tailings stream. Exemplary tailings include, but are not limited to, tailings from oil mining, coal mining, copper mining, gold mining, and mineral processing, such as, for example, processing of phosphate, diamond, gold, mineral sands, zinc, lead, copper, silver, uranium, nickel, iron ore, coal, oil sands, and/or red mud. Exemplary tailings for the present application include tailings from the processing of oil sands. While many of the embodiments are described with reference to oil sands tailings, it is understood that the embodiments, including compositions, processes, and methods, are not limited to applications in oil sands tailings, but also can be applied to various other tailings. The term “tailings” is meant to be inclusive of but not limited to any of the types of tailings discussed herein, for example, process oil sand tailings, in-process tailings, oil sands tailings, and the like.

The terms “oil sands tailings”, “oil sands tailings stream”, “oil sands process tailings”, or, “process oil sand tailings” generally refer to tailings that may be generated as bitumen is extracted from oil sands. Oil sands tailings are generally a mixture of water, sand, fine silts, clay, residual bitumen and lighter hydrocarbons, inorganic salts and water-soluble organic compounds. In tar sand processing, tailings may comprise the whole tar sand ore and any net additions of process water less the recovered bitumen.

The term, “sand” generally may refer to mineral fractions that may comprise a particle diameter greater than 44 microns.

The term “fines” generally may refer to mineral fractions that may comprise a particle diameter less than 44 microns.

The terms, “total solids” or “total suspended solids” are used Interchangeably herein and generally refer the total amount or weight of suspended solids contained in oil sands or other sands comprising dispersion. “Total solids” or “total suspended solids” generally does not include dissolved solids.

The term “clay” generally may refer to materials having a particle size of less than 2 micrometers which comprise mixtures of fine-grained clay minerals, typically hydrous aluminum silicates with variable amounts of other metals, and clay-sized crystals of other minerals such as quartz, carbonate, and metal oxides. Common clays found in oil sands include illite, kaolinite, and montmorillonite. Less common clays include chlorite and vermiculite.

The term “tailings pond” generally refers to engineered dam and dyke facilities used for storage of tailings materials. After waste material is sent to the tailing pond, the sand and clays begin to settle quickly to the bottom; however, the fine solids such as the clays and silts create a floating layer below the surface of the water. Removal of these fine solids is necessary before the process water may be recycled and used again in the mining operation.

As used herein, “fluid fine tailings” or “FFT” may comprise a liquid suspension of oil sand fines in water with a solids content greater than 2%.

The term “mature fine tailings” (“MFT”) generally may refer to fine tailings that may comprise a solids content of about 30-35%, and that generally may comprise almost entirely solids<44 microns. MFT generally may behave as a fluid-like colloidal material. MFT may comprise FFT with a low sand to fines ratio (“SFR”), i.e., generally less than about 0.3, and a solids content that may be generally greater than about 30%.

The term “coagulant” generally may refer to an agent that may typically destabilize colloidal dispersion s to facilitate coagulation, a process of agglomerating colloidal particles into larger particles. Coagulants are added to facilitate removal of suspended solids for process streams, thereby reducing turbidity of the aqueous fraction.

The term “flocculant” may generally refer to a reagent that may bridge neutralized or coagulated particles into larger agglomerates, typically resulting in more efficient settling. Flocculation process generally involves addition of a flocculant followed by mixing to facilitate collisions between particles, allowing for the destabilized particles to agglomerate into larger particles that can be removed by gravity through sedimentation or by other means, e.g., centrifugation, filtration.

The terms “polymer” or “polymeric additives” and similar terms are used in their ordinary sense as understood by one skilled in the art, and thus may be used herein to refer to or describe a large molecule (or group of such molecules) that may comprise recurring units. Polymers may be formed in various ways, including by polymerizing monomers and/or by chemically modifying one or more recurring units of a precursor polymer. Unless otherwise specified, a polymer may comprise a “homopolymer” that may comprise substantially identical recurring units that may be formed by, for example, polymerizing, a particular monomer. Unless otherwise specified, a polymer may also comprise a “copolymer” that may comprise two or more different recurring units that may be formed by, for example, copolymerizing, two or more different monomers, and/or by chemically modifying one or more recurring units of a precursor polymer. Unless otherwise specified, a polymer or copolymer may also comprise a “terpolymer” which generally refers to a polymer that comprises three or more different recurring units. Any one of the one or more polymers discussed herein may be used in any applicable process, for example, as a flocculant.

The terms “aqueous colloidal suspension” or “aqueous colloidal dispersion”, or “aqueous solid dispersion stream” generally refer to a heterogeneous mixture of a fluid that contains solid particles, wherein the solid particles, often termed “colloid” forms a phase separated, mixture in which one substance of microscopically dispersed insoluble or soluble particles is suspended throughout another substance. The colloidal dispersion has a dispersed phase (the suspended particles) and a continuous phase (the medium of suspension) that arise by phase separation. Typically, colloids do not completely settle or take a long time to settle completely into two separated layers. Exemplary aqueous colloidal dispersions for the present application include fine solids, such as the clays and silts, from tailings dispersed in water with trace bitumen from the processing of oil sands.

The term “solid-liquid separation” herein refers to any industrial process by which a process feed from any type of industrial process, for example, an oil or gas extraction or recovery process, tailings treatment process, or any portion thereof comprising liquids and solids is treated to separate water out and produce a solid material or cake. Exemplary solid-liquid separation processes for the current application include centrifugation, thickening, filtration, hydrocyclone, inline flocculation, and/or gravity sedimentation.

The terms “centrifuge feed” or “feed” refers to a process stream from any type of industrial process, for example, an oil or gas extraction or recovery process, tailings treatment process, or any portion thereof that is directed into a dewatering step, which separates water out and produces a solid material or cake. An exemplary feed for the current application includes a mixture of FFT and/or MFT which has been dredged from a tailings pond and fed into a dewatering step after the addition of coagulants and/or flocculants for dewatering.

The terms “centrate”, “centrate stream” “release water” or “reject water” refer to the liquid phase portion of a process stream from any type of industrial process, for example, an oil or gas extraction or recovery process, tailings treatment process, or any portion thereof that has been subjected to a dewatering step. An exemplary release water for the current application comprises the liquid phase of material that has been dredged from an oil sands tailings pond, subjected to the addition of coagulants and/or flocculants, and fed through a dewatering step, producing an aqueous release water and a clay material that may have the consistency of a mud cake.

The terms “total solids”, “total suspended solids”, and “suspended solids” % Solids” refer generally to a total quantity measurement of solid material per unit volume of liquid. This is in contrast with “turbidity”, which is an optical measure of liquid clarity based on scattering and/or attenuation of light passed through a sample as it interacts with suspended solids.

The term “online” herein means real time detection and/or control of a parameter during an industrial process, e.g., an oil sands treatment method. This includes embodiments where the sensor is not physically connected to a computer, e.g., the data is collected real time and stored on sensor memory cards and extracted later for analysis. Also, “online” includes embodiments where a sensor provides for real time detection and/or the control of a process parameter such as by connection to another computer or to a network.

The terms “real time detecting” or “real time monitoring” refer generally to a system in which detection of a phenomena within a sample occurs rapidly and input data is processed and is available virtually immediately for visualization and feedback with little lag time between the actual event and said visualization and feedback. Exemplary processes involving real time monitoring for the current application comprise the use microwave-based sensors for real-time monitoring of total suspended solids in oil sands tailings streams (e.g., feed to centrifuge, centrate, filtrate, filter press, or other process stream). The output signals from the sensor are entered into dosing programs to adjust polymer dosage and maintain total solids within specified limits.

“Controller/Sensor” herein refers to a system that uses computers/networks to monitor various sensors and keep track of what is happening in processes that involve the use of proprietary chemicals at customer plants such as temperature, pH, pressure, particle size distribution, the speed or velocity of the influx or efflux of the aqueous colloidal dispersion through the system, solid-liquid separation rate, dosage of one or more chemicals, amount of free or dissolved air in the sample, or any combination of the foregoing.

### Detailed Description of the Invention

In many conventional oil sands tailings applications, the process streams are monitored with manual measurements on a semi-regular basis. Without the ability to have consistent measurements in real time, it is difficult to properly dose chemicals such as coagulants and flocculants. This results in processes which have on-spec performance a lower percentage of the time than if live data was available through efficient sensors.

Utilizing online measurements to get consistent measurements of solids content of oil sands tailings in real time has been challenging due the nature of process streams which pose challenges for typical sensors utilized in water treatment/process industry like e.g. optical turbidity/TSS (Total Suspended Solids) sensors. Essentially, the high concentration of suspended solids or particles in oil sands tailings results in the composition possessing a turbidity or opaqueness that precludes the use of typical sensors utilized in water treatment processes.

In the current invention, it was surprisingly found that a microwave-based sensor can reliably and accurately be used for real time monitoring total solids from either centrate or feed to centrifuge. As shown infra, it has been demonstrated that solid content measurements obtained using microwave-based sensors are consistent with the values obtained by other detection methods which cannot be used in real time.

The sensor principal is based on change of high frequency wave speed in different media, e.g. the wave goes through water much faster than water containing solids or particles. The changes in speed is directly correlated with the amount of solids in measured media.

As shown infra, the performance of the microwave sensor over time has been validated with more than five centrate samples with different total solids. Also, as further shown infra, parallel total solid measurement has verified the results from microwave sensor. These results indicated good agreement between real time data collected from the sensor and lab measurement. The sensor also accurately and quickly responded to changes in total solids when the sample with lower total solids changed to a sample with higher total solids.

Based on these observations, it has been demonstrated that the output signals from microwave sensors may be used in order to determine whether one or more parameters should be adjusted during industrial process wherein oil sands tailings are being treated. Moreover, while the use of microwave sensors is especially preferred for use in detecting solid content in oil sands tailings, it is expected that microwave sensors may also be used on other industrial processes and systems that are used to treat aqueous colloidal dispersions in order to separate solids from water contained therein. Such methods are especially desired because of the scarcity of water and the desire to reuse the water used in such industrial methods.

Some of the world's largest deposits of oil are located in oil sands formations. Oil sands are comprised of a matrix of loosely consolidated or unconsolidated inorganic solid particulate materials such as sand and clay permeated with oil and water. The oil present in a large proportion of oil sands is viscous bitumen or heavy oil.

Bitumen present in oil sands located within 100 meters of the earth's surface is typically recovered and produced by surface mining the oil sands and then extracting the bitumen from the mined oil sands ore. The oil sands are mined by digging the oil sands from the earth, then transporting the unearthed oil sands ore to a bitumen extraction facility. Bitumen is extracted from the oil sands ore in the extraction facility by crushing the oil sands ore into particulates, mixing the crushed oil sands with an extractant, capturing the bitumen in the extractant, and separating the resulting bitumen containing extract from the inorganic solid particulates of the oil sand.

The most common method of extracting bitumen from mined oil sands ore involves separating the bitumen from inorganic solid particulate material in the oil sands using hot water containing an alkali as the extractant. Hot water, caustic soda, and the mined oil sands ore are mixed into a slurry, and the bitumen is allowed to float to the surface of the slurry where it forms a froth. The bitumen froth is then separated from the inorganic Solid particulate material. Clean oil is produced from the separated bitumen froth by treating the froth to remove water and mineral fines.

Once the bitumen has been removed, a mixture of water, sand, clay, silt, residual bitumen, and persistent amounts of toxic soluble organic compounds that originate from the extraction process, nonlimiting examples of which include, for example, carboxylates, sulfonates and naphthenates is left over. This mixture is referred to as tailings. Water that has been sufficiently separated, purified, and recovered from the tailings may be recycled for reuse in the mining process. This process, however, poses significant difficulty, due to substantial quantities of mineral fines that are not separated from the water with the bulk of the inorganic particulates.

The tailings may comprise a colloidal sludge suspension comprising clay minerals and/or metal oxides/hydroxides. In exemplary embodiments, the tailings stream may comprise water and solids. Tailings generally comprise mineral solids having a variety of particle sizes. Mineral fractions with a particle diameter greater than 44 microns may be referred to as “coarse” particles, or “sand”. Mineral fractions with a particle diameter less than 44 microns may be referred to as “fines” and may essentially be comprised of silica and silicates and clays that may be easily suspended in the water. Ultrafine solids (<2 μm) may also be present in the tailings stream and may be primarily composed of clays. The tailings may include but are not limited to including one or more of the coarse particles, fine tailings, MFT, FFT, or ultrafine solids.

These fines are suspended in the water and are not easily dewatered by conventional mechanical solid/liquid separation techniques such as filtration and centrifugation. Therefore, the mineral fines are separated from the water by placing the water containing the mineral fines in tailings ponds to allow the mineral fines to settle out from the water. Such tailings ponds are undesirable and have become a significant environmental issue. The fresh fine tailings suspension is typically 85% water and 15% fine particles by weight. Dewatering of fine tailings occurs very slowly. When first discharged in the pond, the very low-density material is referred to as thin fine tailings. After a few years when the fine tailings have reached a solids content of about 30-35 wt %, they are sometimes referred to as mature fine tailings (MFT). It may take up to 150 years for MFT to consolidate by gravity to the point when the sediments become trafficable and it is possible to reclaim the land occupied by the tailings pond.

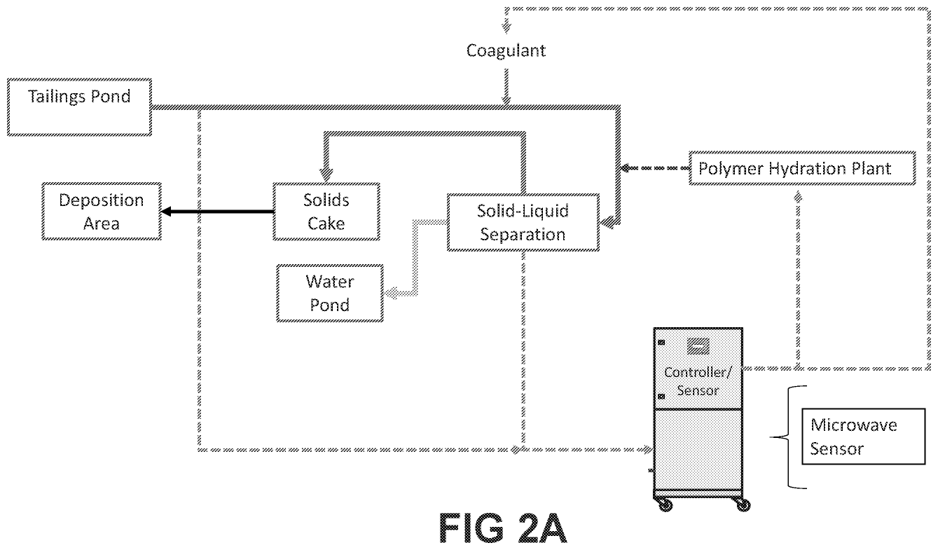

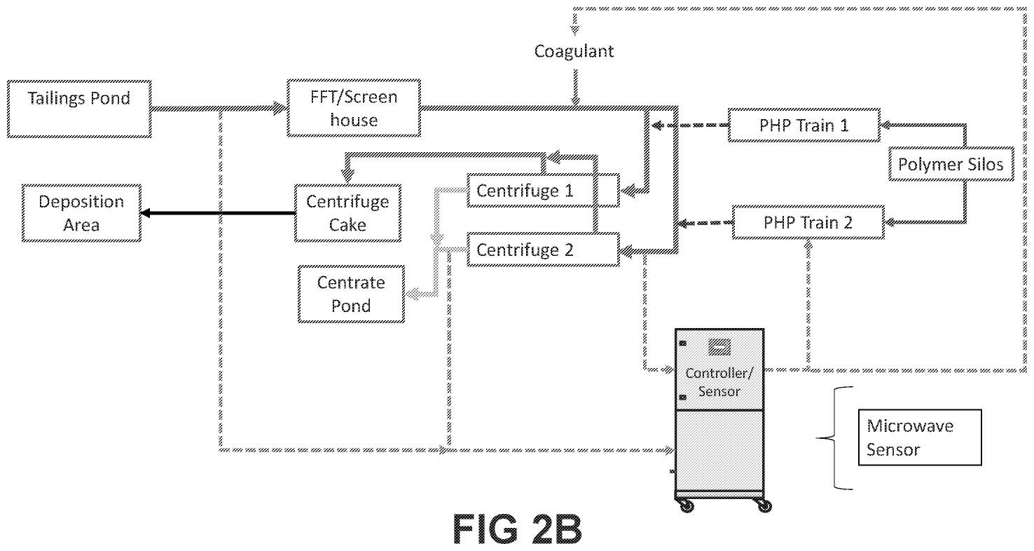

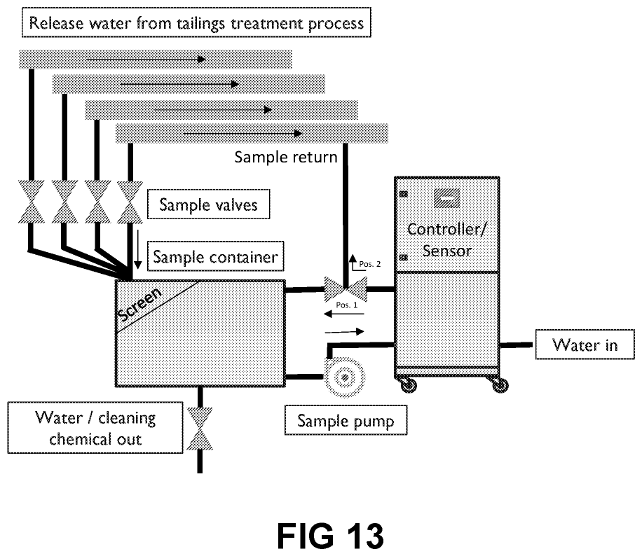

To expedite the removal of MFT, FFT, and ultrafine solids from oil sands tailings, material from the upper layers of tailings ponds may be dredged and fed via pipeline to a treatment facility. In some instances, treatment of mineral or oil sands tailings streams may generally comprise the use of chemicals and polymers including coagulants and/or flocculants to facilitate the agglomeration of MFT, FFT, or ultrafine solids into larger particles, followed by separation of the agglomerated particles by any number of means known to those skilled in the art, e.g., centrifugation, filtration, thickeners. In some embodiments the dredged material may be first passed through a filter or screen to remove larger particulates and clumps of bitumen, prior to addition of coagulants or flocculants, as shown in FIG. 2B or FIG. 13.

In some instances, coagulants, which comprise agents that may typically destabilize colloidal dispersions to facilitate coagulation, a process of agglomerating colloidal particles into larger particles, may be added to the tailings. Exemplary coagulants include e.g., solid coagulants such as metal-based coagulants, e.g., aluminum and iron or other metal-based coagulants, polymeric coagulants, or blends of any of the foregoing. Also, coagulants include gaseous materials, e.g., carbon dioxide, and other coagulants, e.g., those used in thickeners for oil sands tailings treatment. Accordingly, it should be understood that coagulants include any coagulant conventionally used in industrial methods, and particularly those used in oil sands tailings treatment methods.

More particularly, exemplary coagulants may comprise iron-based coagulants, such as ferrous chloride, and/or ferric chloride. Additional examples of iron-based coagulants may include, but are not limited to including ferrous chloride, ferric chloride, ferrous sulfate, ferric sulfate, and/or polyferric sulphate. Other coagulants may be added in addition to an iron-based coagulant, and said other coagulants may comprise but are not limited to comprising inorganic coagulants such as aluminum sulfate (“ALS”) and other metal sulfates and gypsum, organic coagulants such as polyamines and polyDADMACs, and other inorganic and organic coagulants known in the art. In some embodiments, the coagulant may comprise a combination or mixture of one or more iron-based coagulants with one or more other coagulants, e.g., one or more organic coagulants and/or with one or more inorganic coagulants. In some embodiments, said other coagulant may comprise a poly(diallyldimethyl ammonium chloride) (“polyDADMAC”) compound; an epipolyamine compound; a polymer that may comprise one or more quaternary ammonium groups, such as acryloyloxyethyltrimethylammonium chloride, methacryloyloxyethyltrimethylammonium chloride, methacrylamidopropyltrimethylammonium chloride, acrylamidopropyltrimethylammonium chloride; or a mixture thereof. In some embodiments, one or more inorganic coagulants may be added to the tailings stream in addition to one or more iron-based coagulants. An inorganic coagulant may, for example, reduce, neutralize or invert electrical repulsions between particles. Said inorganic coagulants may comprise but are not limited to inorganic salts such as aluminum chloride, aluminum sulfate, aluminum chlorohydrate, polyaluminum chloride, polyaluminum silica sulfate, lime, calcium chloride, calcium sulfate, magnesium chloride, sodium aluminate, various commercially available aluminum salt coagulants, or combinations thereof. In some embodiments, the coagulant may comprise a combination or mixture of one or more of any of the above or other coagulants.

In some instances, treatment of tailings streams may generally comprise the use of flocculants. Flocculants, or flocculating agents, are chemicals that promote flocculation by causing colloids and other suspended particles in liquids to aggregate, thereby forming a floc. Flocculants are generally used in water treatment processes to improve the sedimentation or filterability of small particles. For example, flocculants are used in water treatment processes to improve the sedimentation or filterability of small particles. Flocculants that have been used in treatments for dewatering mineral tailings and oil sands tailings include polyacrylamide polymer flocculants. Among synthetic polymers, those commonly used comprise poly(ethylene oxide) in the nonionic category, poly(diallyldimethylammoniumchloride) (polyDADMAC) in the cationic category, and polyacrylamide (PAM) and poly(styrenic sulfonic acid) in the anionic category. Acrylamide-based polymers, such as cationic emulsion polyacrylamide, are widely used as flocculants in wastewater treatment, and anionic dry polyacrylamides are widely used as flocculants in oil sands tailings treatment.

In some embodiments, the polymer flocculant can be a homopolymer or a copolymer. The term “copolymer” refers to any polymer having more than one type of monomer and may include, for example, terpolymers. Preferably, the copolymer includes two types of monomers. Preferably, the copolymer is a random copolymer.

The monomers of the homopolymer or copolymer may be selected from the group consisting of non-ionic monomers, anionic monomers, and cationic monomers.

In some embodiments, the non-ionic monomer is selected from the group consisting of acrylamide and methacrylamide. Preferably, the non-ionic monomer is acrylamide.

In some embodiments, the anionic monomer is selected from the group consisting of acrylic acid, methacrylic acid, itaconic acid, crotonic acid, fumaric acid, and Acrylamide tertiary butyl sulfonic acid (also known as Acrylamide t-butyl sulfonic acid, N-t-butyl acrylamide sulfonic acid, 2-methylpropane-2-sulfonic acid; prop-2-enamide or ATBS*). Preferably the anionic monomer is acrylic acid or ATBS.

In some embodiments, the cationic monomer is dimethylaminoethylacrylate-methyl chloride (Q9).

When the polymer includes an anionic or cationic monomer, the polymer may further comprise one or more counterions. For example, when the polymer includes an anionic monomer, the counterion may be sodium, calcium or magnesium, preferably sodium or calcium.

Due to the volume of polyacrylamide consumed for mineral or oil sands tailings, dry polyacrylamide (DPAM) is commonly used instead of solution or emulsion polymers. DPAMs typically have standard viscosities (SV) in the range of 2.5-6.5 cP. In some exemplary embodiments DPAMs herein may have standard viscosities (SV) in the range of 2.9-3.2 cP. In mineral or oil sands tailings applications, it has been found that lower molecular weight (MW) products may have the potential to produce flocs with better dewaterability. While higher molecular weight products can provide flocculation, they can be more difficult to mix into the tailings and have a greater tendency to hold water.

During treatment of tailings, the flocculating agent is added to the tailings substrate and flocs, which comprise solid particulate material, are allowed to form. According to the embodiments, the formed flocs may be separated from the aqueous phase of the tailings stream. Separating the flocculated solids from the tailings stream may be accomplished by any means known to those skilled in the art. Exemplary separation methods include but are not limited to centrifuges, hydrocyclones, decantation, filtration, thickeners, or another mechanical and non-mechanical separation methods, e.g., non-mechanical separation methods used in end pit lakes, thin-lift deposition, and deep deposits where no equipment is used to accelerate dewatering of the tailings.

Centrifuges use centrifugal force to separate water out of MFT. By spinning the mixture in a large cylindrical vessel at high speeds (between 1,200 and 1,800 rotations per minute (rpm)), the water is forced from the tailings mixture. Dewatering of tailings by centrifugation typically involves a process in which a mixture of FFT, MFT, and/or ultrafines, which has been dredged from a tailings pond, subjected to the addition of coagulants and/or flocculants, and fed through a centrifuge for dewatering, producing an aqueous centrate and a clay material that may have the consistency of a mud cake.

Adjusting the formulation and dosage of coagulant and/or polymeric flocculant is often necessary to achieve a centrate with desired properties, e.g., total suspended solids, etc. While in theory this adjustment can be used to formulate most effective polymer flocculant combinations and dosages, in practice polymer addition processes may have certain operational constrictions which preclude or impede such adjustments. For example, mineral tailings and oil sands tailings have a range of density and clay content, that can vary over time, and/or by location or other conditions. This variability may present challenges to obtain consistent chemical treatment results. Small changes in the properties of the tailings substrate can change (e.g. reduce) the effectiveness of the chemical treatment. An embodiment of the current invention utilizes real-time monitoring of centrifuge feed by a microwave-based sensor. An “optimal” chemical treatment may be formulated, to handle tailings having a specific subset of these properties.

In particular the subject real time detection methods may be used to prevent polymer overdosing during oil sand tails treatment methods or other industrial processes. This is beneficial as during polymer overdosing the cake solids may decrease because the excess polymer traps more water in the cake and hinders compaction. Also, preventing polymer overdosing is beneficial as excess polymer may accumulate in the release water, which could impede or have a negative impact during water reuse.

After exiting the centrifuge, the centrate or release water may be meet all required specifications (e.g., total suspended solids<3%, etc.) to be either discharged to a water pond or recycled for use in subsequent mining operations. As noted previously, “reject water”, “release water” and “centrate” herein are used interchangeably herein and refer to the aqueous phase of any process stream, the physical properties of which (e.g. total suspended solids) do not conform to predetermined specifications. Reject or release water may generally require further expensive and time-consuming processing or treatment to meet specifications. An embodiment of the current invention utilizes real-time monitoring of centrate or reject water by a microwave-based sensor. For example, release waters may include filtrates or overflow waters produced or used in applications such as filtration, thickeners, etc. As noted, the output signals from the microwave sensor may be used in order to determine whether one or more parameters should be adjusted during the industrial process wherein oil sands tailings are being treated. A particular application comprises using the detected solid content amount in order to determine whether the dosage amount of chemicals used in the treatment process, e.g., polymers such as coagulants and flocculants used therein should be modified. For example the detected solids amount may be entered into a dosing program which is then used to adjust the amount of the chemical, e.g., a polymer dosage and/or to retain total solids of centrate within a certain limit that is commercially desirable.

In exemplary usages the treated oil sand tailings contains bitumen which may form a clump when polymer is added in slurry. The clump could block or restrict flow in flow through sensor, and consequently an erroneous signal could be obtained. To avoid this potential problem, the invention contemplates the addition of a prefilter (strainer) in the system used, particularly to have a prefilter (strainer) on centrate stream prior to sensor.

Moreover, the invention is not limited to the use of microwave sensors for detecting total solid content in the centrifuge. For example, microwave sensors should also be useful for monitoring and measuring total solids in other equipment such as thickeners and filter presses which are used in oil sand tailing treatment as well as other industrial processes used to treat aqueous colloidal dispersions in order to separate solids from water contained therein.

The subject microwave detection methods should provide substantial benefits. For example, it is expected that this real time detection will permit such industrial methods to be conducted more rapidly because adjusting parameters real time such as polymer dosages among others should increase process efficiency.

Also, it is expected that the subject microwave detection methods should permit the usage of reduced amounts of chemicals such as polymer coagulants and flocculants.

Further, it is expected that the subject microwave detection methods should permit the removal of greater amounts of solid from the treated aqueous colloidal dispersion, e.g., oil sand tailings.

Moreover, it is expected that the subject microwave detection methods should permit the removal of greater amounts of water and/or greater purity from the treated aqueous colloidal dispersion, e.g., oil sand tailings.

Still further, it is expected that the subject microwave detection methods should permit the equipment to be less subject to breakdown because of the ability to maintain solid content level within desired or acceptable levels.

Moreover, the invention contemplates processes conducted under pressure (e.g., 0.2-5 bar, typically 1-3 bar and more typically 1.5 to 2.0 bar) through the sensor to keep free air dissolved in the sample and thereby prevent or inhibit air bubble formation. This should preclude such air bubbles from potentially interfering and causing the microwave sensor to stop working and/or result in erroneous microwave measurements especially in processes that detect lower total solids amounts.

Moreover, the invention contemplates microwave detection methods that comprise measurement systems whereby the sensor can be kept clean without unnecessary manual work. Particularly, the invention contemplates systems that are equipped with suitable automatic cleaning systems, e.g., water flushing system or chemical cleaning setup for sensor.

Having described the invention in detail the invention is further described in the following examples.

### EXAMPLES

The following examples are presented for illustrative purposes only and are not intended to be limiting.

### Example 1: Measurement of Total Suspended Solids in Centrate Sample with Turbidimeter

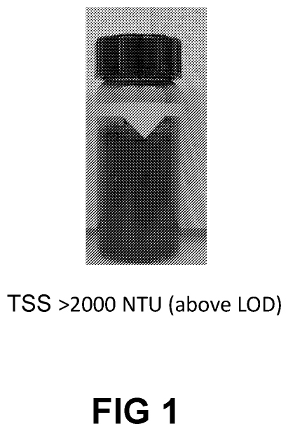

A lab turbidimeter was used for measuring total suspended solids (TSS) from centrate samples taken from oil sands tailings process streams. The TSS values were above detection limit of turbidimeter (>2000 NTU). This was due to the opaque appearance of the samples, which prevented light transmission. These results indicate that measuring methods based on scattering/transmission of light, as with a turbidimeter, are not suitable for accurate real-time monitoring of total suspended solids in oil sands tailings streams including. FIG. 1 presents an exemplary image of a centrate sample, which was analyzed for the present example.

### Example 2: Flow Diagram of Oil Tailing Dewatering Process by Centrifuge Showing Exemplary Location of the Sensor and Controller/Sensor in Process

Exemplary flow charts of material flow (solid lines) and data flow (dashed lines) in industrial processes for treating tailings streams are shown in FIG. 2.

Each chart depicts of one of many possible methods for using a microwave-based sensor for real-time monitoring of total suspended solids in oil sands tailings streams including, but not limited to, feed and/or exhaust water from solid-liquid separation and total solids in centrate and/or feed to centrifuges. One exemplary flow chart depicts a process of oil sands tailings dewatering by centrifuge wherein the microwave sensor and Controller/Sensor is located within a control box to (i) monitor total suspended solids in the centrifuge feeding line and centrate lines, (ii) create a data log of sensor output, (iii) enter output signals from the sensor into dosing programs to adjust dosage of polymeric additives from polymer hydration plant (PHP Trains 1 and 2) to maintain total solids of the centrate within specified limits, and (iv) visualize data and dosing on a dashboard or graphical user interface.

### Example 3: Diagram of Microwave Sensor Used for Measuring Solids in Real-Time

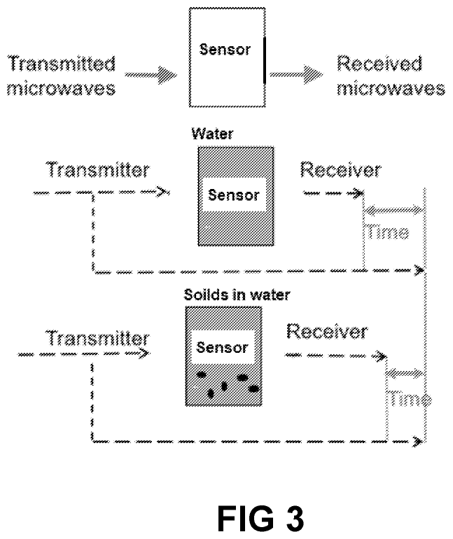

An exemplary microwave-based flow-through sensor was integrated, along with Controller/Sensor, into the oil sands tailings dewatering process. Real-time measurement of total solids in feed and exhaust water was carried out with a flow-through sensor based on microwave technology as shown in FIG. 3. Microwaves are high frequency radio waves (1.5-2.5 GHz) like radars and mobile phone with power intensity of 100 mW. The microwave travels between transmitter, through the sample medium, and then to receiver antennas in the sensor. The speed depends on the medium through which the wave travels (e.g., relative speed in air is 1, in water 0.1, and in water containing fiber or filler 0.4-0.6). The sensor uses a single point calibration and can, for example, measure total solids in process streams from 0-50% over wide ranges of temperature (0-100° C.) and pH (2.5-11.5).

### Example 4: Test Rig for Evaluation of Microwave Sensing of Total Solids in Real-Time

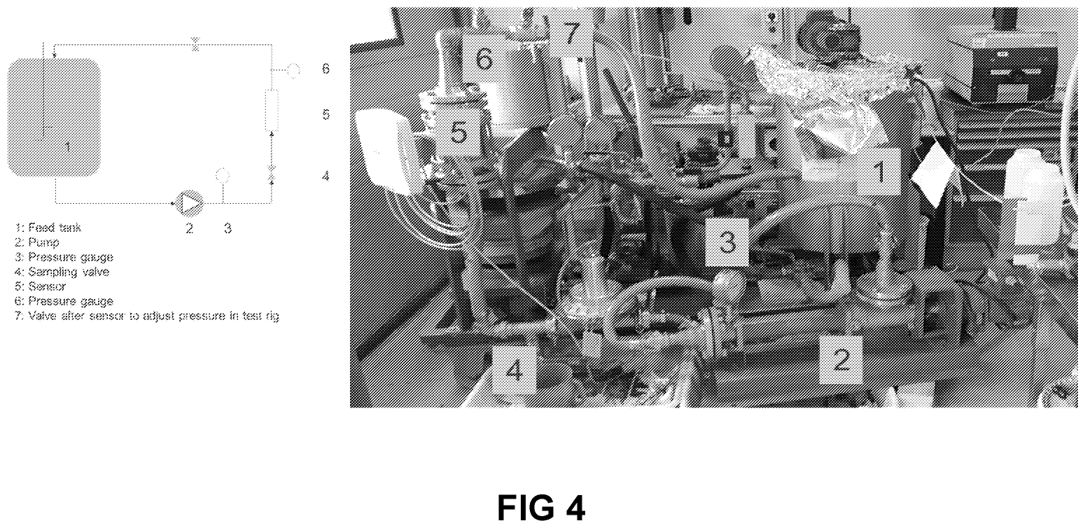

Evaluation of a microwave sensor for real-time monitoring of total solids in centrate samples was carried out in a test rig built in Kemira Espoo R&D. An example image and diagram of the test rig is shown in FIG. 4. Centrate samples from oil sands tailings process streams were received, poured into the feed tank (FIG. 4, number 1), and then circulated via pump (FIG. 4, number 2) through a microwave sensor (FIG. 4, number 5) described in detail in Example 3. The real-time signal from microwave sensor was logged in Controller/Sensor. Data from the microwave sensor indicating total solids is denoted as Dry solid sensor (%), Total Solids (real time), Total solid (sensor), or Solids % (sensor). Samples were also manually collected from either sampling valve (FIG. 4, number 4) or from the return pipe to the feed tank (FIG. 4, number 1). Total solids from these samples were measured with a halogen dryer at 105° C. Data from these parallel dry solids measurements was logged in Controller/Sensor and is denoted as Total solids (lab), Dry solid %, Solids % (lab), or Dry solids quick test. These parallel dry solids measurements were performed to verify accuracy of the results from the microwave sensor.

### Example 5: Verification of the Accuracy of Microwave Sensing of Total Solids in Real-Time

In the following example, parallel total solids measurements were performed to verify accuracy of the results from the microwave sensor. Five different centrate samples from oil sands tailings process streams were received and the average total dry solids was measured with a halogen dryer at 105° C. Results are shown Table 1. The samples and corresponding data were then used to calibrate the microwave sensor of the test rig described in Example 4.

TABLE 1
Total solids of centrate samples

| Sample | Dry solid, % |
|---|---|
| Clean centrate | 8.50 |
| Dirty centrate | 12.20 |
| Centrate water sample 1 | 0.77 |
| Centrate water sample 2 | 7.36 |
| Centrate water sample 3 | 12.83 |

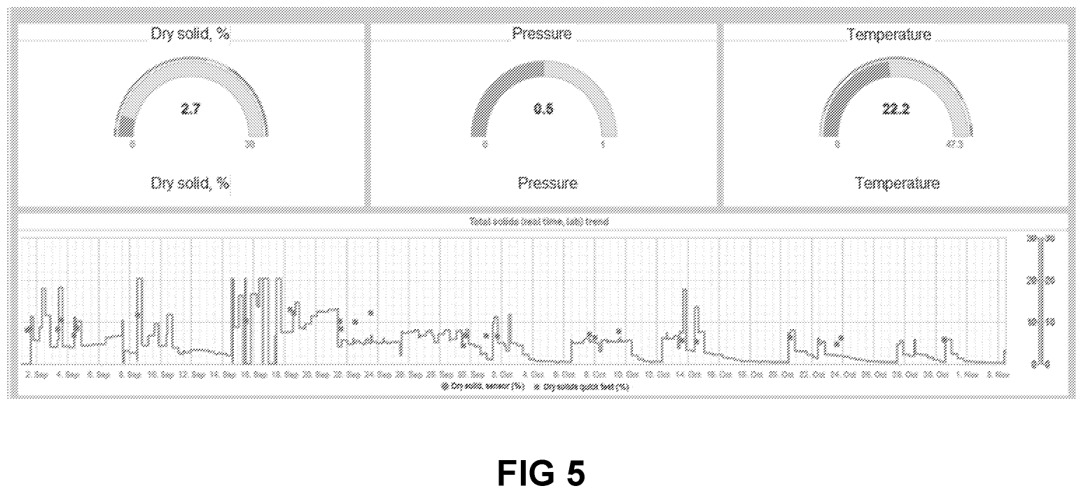

The samples were then fed into the test rig for parallel evaluation of real-time data from the microwave sensor and dry solids data from manually collected samples. The experiment was performed, and data was logged into Controller/Sensor as described in Example 4. A Controller/Sensor dashboard was built to follow results from the measurement. Dry solids (%) from the sensor, temperature, and pressure were visualized in real time. Solids from lab measurements (Dry solids quick test (%)) were added manually. An exemplary snapshot of the Controller/Sensor dashboard built to follow up the sensor performance is shown in FIG. 5.

The results indicated good agreement between real-time data collected from the microwave sensor and lab measurements. The online sensor also responded to changes in total solids when the sample with lower total solids was changed to a sample with higher total solids.

### Example 6: Additional Verification of the Accuracy of Microwave Sensing of Total Solids in Real-Time

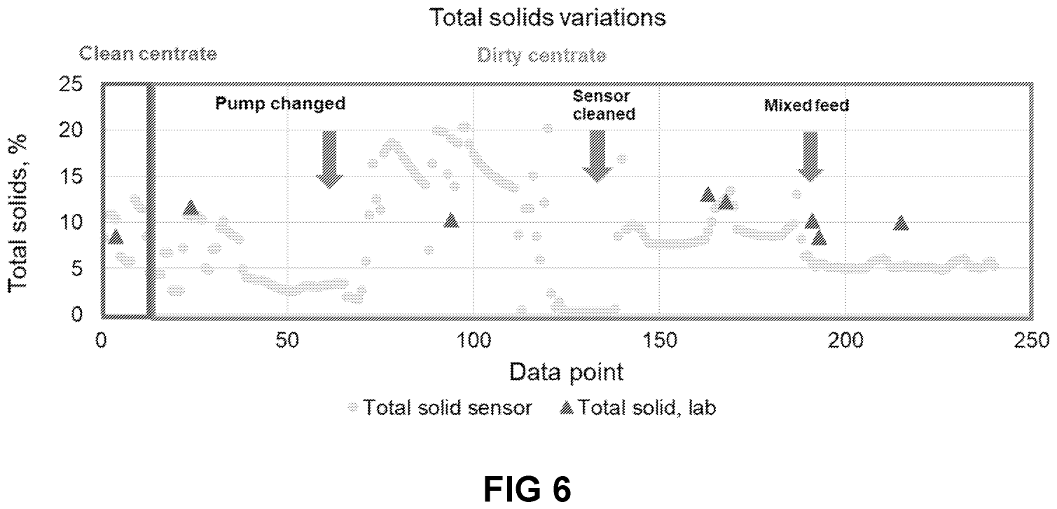

In this example, sensor performance over several days was validated with two additional centrate samples with different total solids contents as described in Example 5. Parallel total solids measurements were performed to verify accuracy of the results from the microwave sensor. Centrates were monitored in real-time for total solids using a microwave-based sensor of the present invention resulting in >200 data points. Samples were also manually removed from the same centrate stream and sent for laboratory analysis of total solids resulting in 8 data points over the same time period. An exemplary graph showing total % solids in the centrate stream determined by (i) real-time microwave sensor monitoring and (ii) laboratory analysis of manual samples over time is shown in FIG. 6.

The results indicated good agreement between real-time data collected from the microwave sensor and lab measurements. The sensor also responded to changes in total solids when the sample with lower total solids changed to a sample with higher total solids. Cleaning of the sensor restored the signal from zero to 10% total solids, indicating a need for in-process automated sensor cleaning.

### Example 7: Correlation Between Microwave Sensor and Lab Measurement

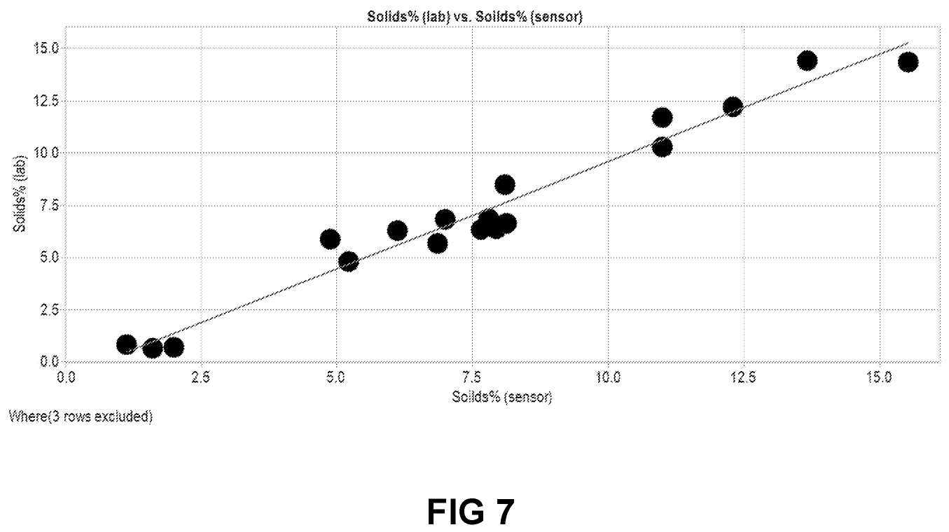

Centrate samples were analyzed in parallel for total solids by (i) real-time microwave sensor monitoring and (ii) laboratory analysis of manual samples according to Example 5. Experimental data was plotted to show correlation between dry solids measured with real-time sensor and lab measurements. A graph of Solids % (lab) as a function of Solids % (sensor) and trend line are shown in FIG. 7.

These results indicate a strong positive linear correlation between Solids % (lab) and Solids % (sensor) providing further proof of concept.

### Example 8: Correlation Between Microwave Sensor and Lab Measurement Over a Range of Temperatures

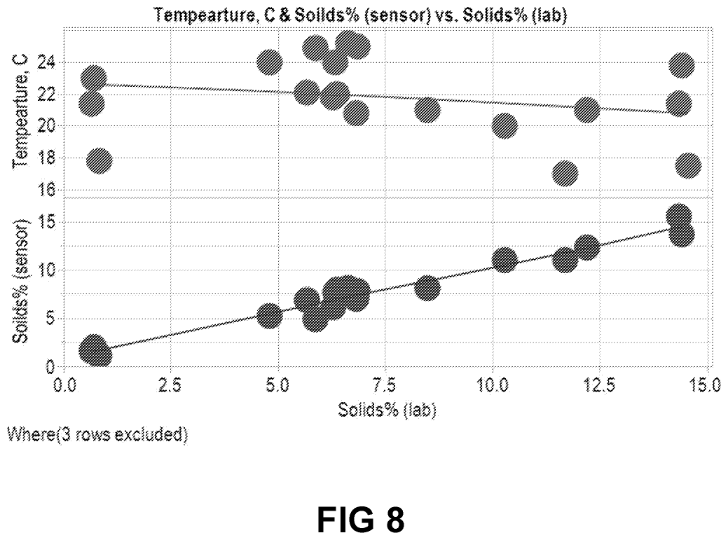

Centrate samples were analyzed in parallel for total solids by (i) real-time microwave sensor monitoring and (ii) laboratory analysis of manual samples according to Example 5. Experimental data collected across a range of temperatures (18 to 24° C.) was plotted to show correlation between dry solids measured with real-time sensor and lab measurements. Stacked graphs of temperature variation during the experiment (top) and Solids % (lab) as a function of Solids % (sensor) with trend line (bottom) are shown in FIG. 8.

The results indicate little influence of temperature variance from 18 to 24° C. on solid measurements. A strong positive linear correlation between Solids % (lab) and Solids % (sensor) was observed across all experimental temperatures.

### Example 9: Correlation Between Microwave Sensor and Lab Measurement Over a Range of Temperatures, Conductivities, and pH Values

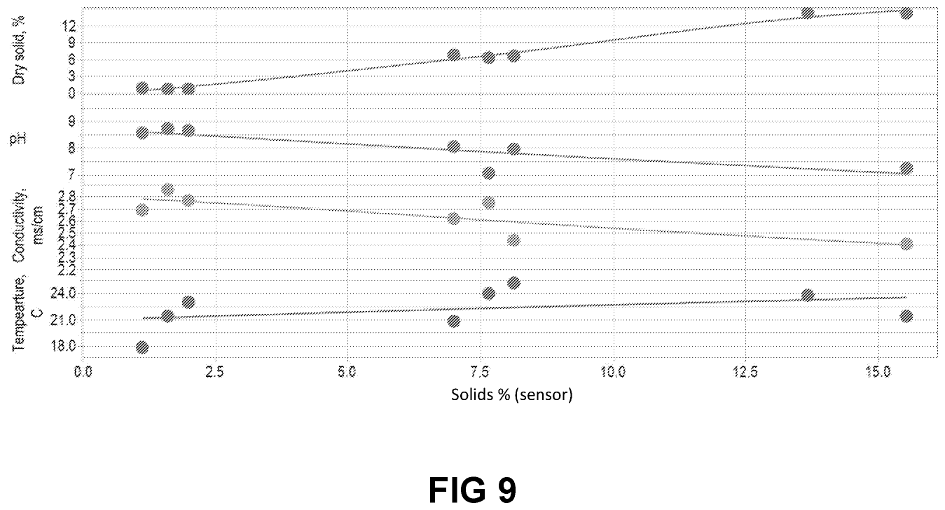

Centrate samples were analyzed in parallel for total solids by (i) real-time microwave sensor monitoring and (ii) laboratory analysis of manual samples according to Example 5. Experimental data collected across a range of temperatures (18 to 25° C.), conductivities (2.4 to 2.9 ms/cm), and pH values (7 to 8.5) was plotted to show correlation between dry solids measured with real-time sensor and lab measurements. Stacked graphs of variation in temperature, conductivity, and pH during the experiment and a graph of Solids % (lab) as a function of Solids % (sensor) with trend lines (bottom) are shown in FIG. 9.

Results indicate little influence of variation in temperature, conductivity, or pH, on solid measurements. A strong positive linear correlation between Solids % (lab) and Solids % (sensor) was observed across all experimental conditions.

### Example 10: Effect of Pressure on the Correlation Between Solids % (Lab) and Solids % (Sensor

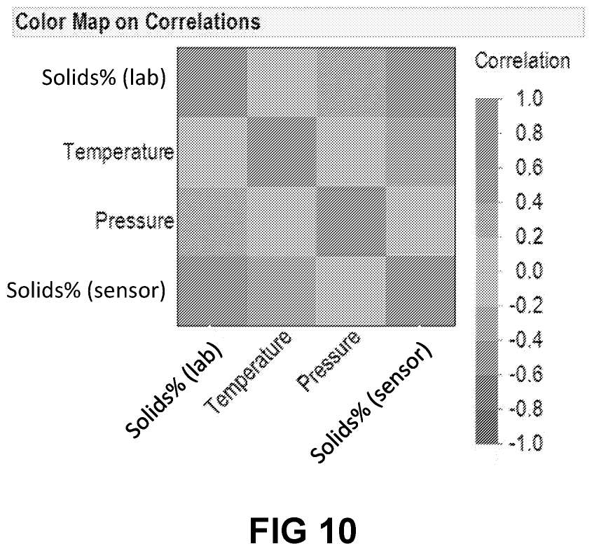

As described in Example 3, the speed of microwaves in air is different from water. During the test trials described in Examples 4-6, it was noticed that foam formed during feed circulation. When dissolved gasses within the circulating feed flow through the microwave sensor, they may be released to form bubbles, which interfere with the signal. This is more obvious when the feed solids are on low range. To keep dissolved gas in the liquid feed, a pump was used to increase pressure to 1.5-2.0 bar to keep free air dissolved in the sample and prevent air bubble formation. Centrate samples were then analyzed in parallel for total solids by (i) real-time microwave sensor monitoring and (ii) laboratory analysis of manual samples according to Example 5. Temperature and pressure data were recorded during the experiment. An exemplary color map of correlation factors from parallel comparison of real-time microwave sensor data (Solids % (sensor)) and lab measurement (Solids % (lab)) across a range of temperatures and pressures is shown in FIG. 10.

Results indicate that Solids % (lab) and Solids % (sensor) have strong positive correlation (0.8-1). Signal from the microwave sensor is negatively correlated with temperature and pressure; however, the correlation is very weak (−0.2), suggesting that the increased pressures have little to no detrimental effect on the measurements.

### Example 11: Correlation Factors Between Solids % (Lab), Temperature, Conductivity. PH, and Solids % (Sensor)

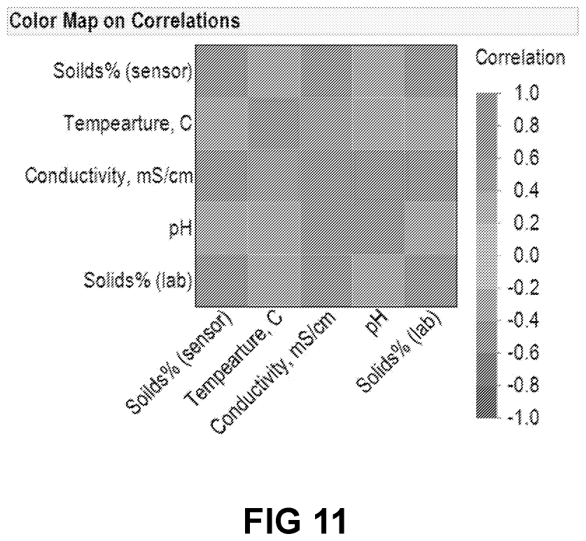

Centrate samples were analyzed in parallel for total solids by (i) real-time microwave sensor monitoring and (ii) laboratory analysis of manual samples according to Example 5. Temperature, conductivity, and pH data were recorded during the experiment. An exemplary color map of correlation factors from parallel comparison of real-time microwave sensor data (Solids % (sensor)) and lab measurement (Solids % (lab)) across a range of temperatures, conductivities, and pH values is shown in FIG. 11.

Results indicate that Solids % (lab) and Solids % (sensor) have strong positive correlation (0.8-1). Variables (pH and temperature) are negatively correlated with signal from the microwave sensor, however, the correlation factor is smaller than 0.5 so it is considered weak. Conductivity was negatively correlated with signal from the microwave sensor (−0.6). This is in line with information provided by the sensor supplier indicating that results may be falsely influenced at high conductivity.

### Example 12: Test Rig and Sensor Blockage

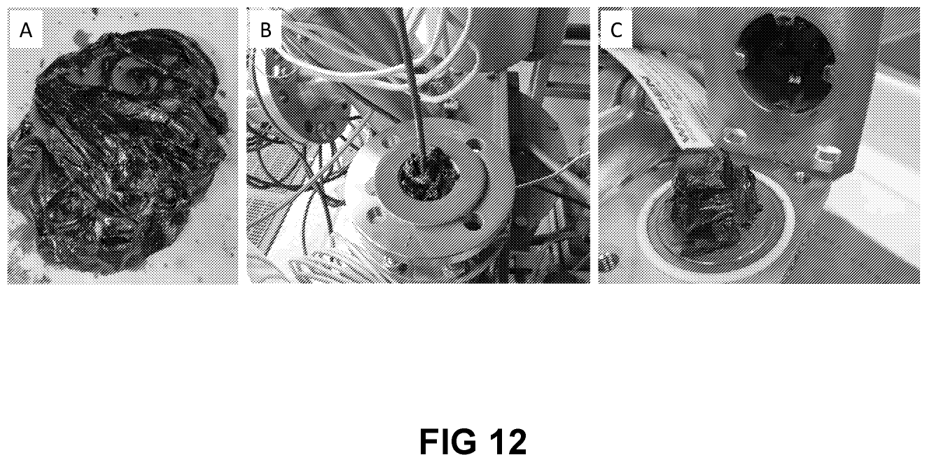

Centrate sample received in the lab contained residual bitumen. When circulated through the test rig as described in Example 4, the bitumen formed a blockage within the sensor and pump. Exemplary images of bitumen blockage in the test rig are shown in FIG. 12.

It can be concluded that, in field applications of the present invention, it may be beneficial to use a strainer (oil trap) to remove residual bitumen prior to the sensor. Another option is to have a cleaning loop in place and clean the sensor frequently.

### Example 13: Flow Diagram of Oil Tailing Dewatering Process

As afore-mentioned, one of many possible methods for using a microwave-based sensor for real-time monitoring of total suspended solids in oil sands tailings streams includes, but is not limited to, measuring total solids in release water from tailings treatment processes, wherein a filtration screen is employed as a pretreatment method to remove large particulates and an automatic cleaning system is employed to allow for extended use without manual cleaning. An exemplary flow chart is shown in FIG. 13.

Having described exemplary embodiments of the invention, the invention is further described in the claims which follow.

What is claimed is:

1. An industrial process for treating an aqueous colloidal dispersion that comprises the following:
a) separating solids from water comprised in an aqueous colloidal dispersion, that comprises water and solids, and
b) detecting and/or monitoring the solids content (suspended solids content) of an aqueous colloidal dispersion in real time with a microwave sensor, optionally wherein said method further includes detecting in real time one or more other parameters with sensors, e.g., (i) pH, (ii) particle size, (iii) temperature, (iv) pressure, (v) solid-liquid separation rate (vi) influx or efflux rate of colloidal dispersion, (vii) amount of free or dissolved air or CO₂ in the detected sample or any combination of the foregoing.

2. The industrial process of claim 1, wherein said real time detection and/or monitoring of the solids content of the aqueous colloidal dispersion with a microwave sensor and/or real time detection and/or monitoring of (i) pH, (ii) particle size, (lii) temperature, (iv) pressure, (v) solid-liquid separation rate (vi) influx or efflux rate of colloidal dispersion, (vii) amount of free or dissolved air or CO₂ in the detected sample or any combination of the foregoing is effected continuously or intermittently as the industrial process proceeds.

3. The industrial process of claim 1 or 2, wherein said process further includes a step c) wherein the detected amount of solids alone or optionally in conjunction with one or more other parameters detected in real time, is used to determine whether one or more parameters should be modified during the process, optionally wherein the detected solids amount is input to a controller where further optionally, based on a dosing algorithm, an output signal is sent to dosing pumps, wherein said other detected parameters optionally include (i) temperature, (ii) pH, (iii) pressure, (iv) the introduction of a chemical or other moiety into the system such as a coagulant, flocculant, biocide, enzyme, or polymer, (v) the adjustment of the dosage of any of the foregoing, (vi) the speed or velocity of the influx or efflux of the aqueous colloidal dispersion through the system, (vii) solid-liquid separation rate (e.g., g-force or rpm), or any combination of the foregoing, wherein optionally said controllers provide for one or more of said parameters to be adjusted based on the detected solids content alone or in association with another detected parameter.

4. The industrial process of claim 1 or 2, wherein the aqueous colloidal dispersion comprises oil sands tailings or another aqueous colloidal dispersion comprising bitumen or other dark solids and/or it comprises an optically turbid aqueous colloidal dispersion comprising dispersed or colloidal solids.

5. The industrial process of any one of claims 1-4, wherein the aqueous colloidal dispersion comprises oil sands tailings, mineral industry tailings, or mining tailings.

6. The industrial process of any one of claims 1-5, wherein
i) said real time detecting and/or monitoring of the solids content of the aqueous colloidal dispersion with a microwave sensor is effected continuously or periodically as the industrial process is conducted;
ii) the amount of detected solids in the aqueous colloidal dispersion, e.g., oil sand tailings composition, is used to adjust the dosage of coagulant or flocculant added to the system, pH, temperature, solid-liquid separation rate, influx or efflux of colloidal dispersion, or any combination thereof;
iii) the amount of detected solids is detected in a centrate, filtrate, overflow, and/or release water from thickeners, drainage applications, thin-lift, or any other industrial process, e.g., an oil sands treatment process;
iv) the amount of detected solids is detected in any equipment used in an industrial process for treating an aqueous colloidal dispersion to separate solids therefrom, e.g., an oil sands treatment process;
v) the amount of detected solids is detected in the feed to the oil sands tailings treatment process, e.g., to the centrifuge, filter press, thickener, inline flocculation or any other industrial process;
vi) the process is run under pressure, optionally 0.2-5 bar, typically 1-3 bar and more typically 1.5 to 2.0 bar;
vii) one or more other parameters are detected in real time, e.g., temperature, pressure, pH, the speed or velocity of the influx or efflux of the aqueous colloidal dispersion through the system, solid-liquid separation rate, e.g., g-force or rpm; dosage of one or more chemicals, amount of free or dissolved air or CO₂ in the sample, (mean or average) particle size of dispersed solids, or any combination of the foregoing, wherein optionally one or more of said parameters are periodically or continuously detected by use of a system that uses computers/networks to monitor in real time via one or more sensors that detect said one or more parameters;
viii) in the process of claim vii), wherein the parameters periodically or continuously detected include any of the following in the oil sands stream or other aqueous solid dispersion: detection of moisture content, detection of pH, detection of elemental composition, detection of sulfur content, detection of iron content, detection of clay amount, detection of magnesium amount, detection of sodium amount, detection of aluminum amount, detection of calcium amount, detection of hydrogen amount, detection of silicon amount, detection of potassium amount, detection of particle size, e.g., average or mean particle size, or any combination of the foregoing;
ix) the process includes the use of one or more gamma detectors or gamma neutron activation analyzers, microwave sensors, pressure sensors, temperature sensors or dissolved air sensors; or
x) any combination of i) to ix).

7. The industrial process of any one of claims 1-6, wherein
i) the detected amount of solids is periodically compared to that of a control sample containing a known solids content as a quality control;
ii) a prefilter or strainer or other removal means is used to remove large particles or clumps optionally comprising bitumen, from the centrate stream, or other aqueous solid dispersion stream prior to the stream being contacted with the microwave sensor;
iii) the system includes an automatic cleaning system, optionally a water flushing system or a chemical cleaning setup for the sensor,
iv) the system Includes a filter cleaning system which optionally removes large particles or clumps optionally comprising bitumen from the filter;
v) the system is schematically depicted in FIG. 2A, 2B, or 13; or
vi) any combination of i) to v).

8. The industrial process of any one of claims 1-7, which
i) permits the usage of reduced amounts of one or more polymers, e.g., flocculants or coagulants used in the industrial process, optionally at least 5, 10, 20, or 30% less compared to processes conducted without real time monitoring of dispersed solid content and optionally based thereon adjustment of one or more process parameters such as dosing, pH, pressure, solid-liquid separation rate, influx or efflux velocity, amount of free or dissolved air or CO₂ in the sample or any combination thereof;
ii) said microwave monitoring of total solids content in real time provides for the separation of more solids from the treated aqueous colloidal dispersion, e.g., oil sands tailings, compared to processes conducted without real time monitoring of dispersed solid content and optionally based thereon the adjustment of one or more process parameters such as dosing, pH, pressure, solid-liquid separation rate, influx or efflux velocity, amount of free or dissolved air or CO₂ in the sample or any combination thereof;
iii) provides for the separation of more water, and/or the recovery of higher purity water, from the treated aqueous colloidal dispersion, e.g., oil sands tailings or other tailings composition, than a process without real time monitoring of dispersed solids and optionally based thereon the adjustment of one or more process parameters such as dosing, pH, pressure, solid-liquid separation rate, influx or efflux velocity, amount of free or dissolved air or CO₂ in the sample or any combination thereof;
iv) optionally provides for the process to be conducted more rapidly than processes conducted without such real time monitoring of solids content and further optionally based thereon the adjustment of one or more process parameters such as dosing, pH, pressure, solid-liquid separation rate, influx or efflux velocity, amount of free or dissolved air or CO₂ in the sample, or any combination thereof;
v) is conducted at pressures or conditions which reduce or preclude air bubble formation; or
vi) any combination of i) to v).

9. An industrial system useful in separating water from solids comprised in an aqueous colloidal dispersion that comprises water and solids, optionally oil sands tailings, wherein the system comprises one or more microwave sensors that detect and/or monitor in real time the solids content of said aqueous colloidal dispersion as said industrial process proceeds.

10. The industrial system of claim 9, which includes a centrifuge, filter press, thickener, hydrocyclone, inline flocculation, thin lift deposition, end pit lake, or combination thereof.

11. The industrial system of claim 9 or 10, which comprises multiple sensors which are optionally connected to computers/networks to determine process parameters in the industrial system in real time, optionally process parameters such as dosing, pH, pressure, solid-liquid separation rate, particle size, influx or efflux velocity, amount of free or dissolved air or CO₂ in the sample or any combination thereof.

12. The industrial system of any one of claims 9-11, which includes one or more sensors that detect one or more of the following in the oil sands stream or other aqueous solid dispersion or the equipment used in the system: moisture content, pH, elemental composition, sulfur content, iron content, clay amount, magnesium amount, sodium amount, aluminum amount, calcium amount, hydrogen amount, silicon amount, potassium amount, particle size distribution, or any combination of the foregoing.

13. The industrial system of any one of claims 9-12, which includes one or more gamma detectors or gamma neutron activation analyzers, microwave sensors, pressure sensors, temperature sensors or dissolved air sensors.

14. The industrial system of any one of claims 9-13, which comprises any or all of the components depicted schematically in FIG. 2 or 13.

15. The industrial system of any one of claims 9-14, wherein a prefilter or strainer or other removal means is comprised therein which is used to remove large particles or clumps from the centrate stream or other aqueous solid dispersion stream prior to the stream being contacted with the microwave sensor.

16. The industrial system of any one of claims 9-15, wherein the system includes an automatic cleaning system, optionally a water flushing system or a chemical cleaning setup for the sensor, preferably one which removes bitumen from the microwave sensor that detects solids.
